(19) United States
(12) Patent Application Publication
(10) Pub. No.: US 2005/0283108 A1
(43) Pub. Date: Dec. 22, 2005

(54) Apparatus and method for non-pharmacological treatment of glaucoma and lowering intraocular pressure

(76) Inventor: James A. Savage, Greensboro, NC (US)

Correspondence Address: BROUSE MCDOWELL LPA, 388 SOUTH MAIN STREET, SUITE 500, AKRON, OH, 44311, US

(21) Appl. No.: 11/149,574

(22) Filed: Jun. 10, 2005

Related U.S. Application Data
(60) Provisional application No. 60/578,487, filed on Jun. 10, 2004.

(51) Int. Cl. A61M 5/00

(52) U.S. Cl. 604/8

(57) ABSTRACT

An apparatus and method for treating glaucoma and lowering IOP is herein disclosed. A method for draining aqueous includes creating an incision in the sclera, opening a scleral flap, and inserting a drainage tube between an associated anterior chamber and at least one aqueous vein. The device has a tube with proximal and distal ends that connects between the anterior chamber and the aqueous veins, collector channels, veins, or distal veins.

This application claims priority to a provisional patent application, with Ser. No. 60/578,487, entitled A METHOD FOR NON-PHARMACOLOGICAL TREATMENT OF GLAUCOMA AND LOWERING INTRAOCULAR PRESSURE, filed Jun. 10, 2004, the contents of which are herein incorporated by reference.

### I. BACKGROUND OF THE INVENTION

A. Field of Invention

This invention relates to an apparatus and method for treating glaucoma, and more particularly to an apparatus and method for non-pharmacological treatment of glaucoma and lowering intraocular pressure.

B. Description of the Related Art

It is known in the art that the treatment of glaucoma consists in lowering the intraocular pressure (IOP) to a level that is tolerable for the optic nerve so that the progression of damage and visual loss is halted.

Glaucoma is a significant public health problem, because glaucoma is a major cause of blindness. The blindness that results from glaucoma involves both central and peripheral (“side”) vision and has a major impact on an individual's ability to lead an independent and productive life.

Glaucoma is an optic neuropathy (a disorder of the optic nerve) that usually occurs in the setting of an elevated intraocular pressure. The pressure within the eye increases and this is associated with changes in the appearance (“cupping”) and function (“blind spots” or “scotomas” in the visual field) of the optic nerve. If the pressure remains high enough for a sufficient period of time, total loss of vision occurs.

High pressure develops in an eye because of an internal fluid imbalance. The eye is a hollow structure that contains a clear fluid called “aqueous humor.” Aqueous humor is formed in the posterior chamber of the eye behind the iris by the ciliary body at a rate of approximately 2.5 microliters per minute. Aqueous humor, which is made at a fairly constant rate, then passes around the lens, through the pupillary opening in the iris and into the anterior chamber of the eye. Once in the anterior chamber, the fluid drains out of the eye through two different routes. The primary pathway for aqueous outflow in humans is through the “canalicular” route through the trabecular meshwork and into Schlemm's canal from which it proceeds into the venous circulation. In the “uveoscleral” route, the fluid percolates between the muscle fibers of the ciliary muscle. This route accounts for approximately ten percent of the aqueous outflow in humans.

The trabecular meshwork and Schlemm's canal are located in the cornea at the junction between the iris and the cornea. This junction or corner is called the “angle.” The trabecular meshwork is a ring of tissue, which is wedge-shaped in cross-section that runs in the internal peripheral cornea around the circumference of the eye. It is composed of collagen beams arranged in a three-dimensional sieve-like structure. The beams are lined with a monolayer of cells called trabecular cells. The spaces between the collagen beams are filled with extracellular substance that is produced by the trabecular cells. These cells also produce enzymes that degrade the extracellular material. Schlemm's canal is adjacent and external to the trabecular meshwork. The outer wall of the trabecular meshwork coincides with the inner wall of Schlemm's canal. Schlemm's canal is a tube-like structure that runs around the circumference of the cornea but may not be perfectly regular and circumferential in its course.

Aqueous humor travels through the spaces between the trabecular beams, across the inner wall of Schlemm's canal and into the canal, through a series of about twenty-five aqueous veins that drain from Schlenun's canal and into collector channels and then into the episcleral venous system. From there the aqueous drains into the systemic venous circulation. In a normal situation, aqueous production is approximately equal to aqueous outflow and intraocular pressure (IOP) remains fairly constant in the 15 to 21 mmHg range. In most cases of glaucoma, the resistance through the canalicular outflow system is abnormally high, resulting in elevated IOP.

In primary open angle glaucoma, which is the most common form of glaucoma, the abnormal resistance is believed to be in the juxtacanalicular tissue along the outer aspect of the trabecular meshwork and the inner wall of Schlemm's canal. It is believed that an abnormal metabolism of the trabecular cells leads to an excessive buildup of extracellular material or a buildup of abnormally “stiff” materials in this area. Histopathology of glaucomatous eyes also demonstrates a collapse of Schlemm's canal. Primary open angle glaucoma accounts for approximately eighty-five percent of all glaucoma in Caucasian and Black populations. Other forms of glaucoma (such as angle closure glaucoma and secondary glaucoma) also involve decreased outflow through the canalicular pathway, but the increased resistance is from other causes such as mechanical blockage, inflammatory debris, cellular blockage, etc.

With the increased resistance to aqueous outflow, the aqueous builds up in the anterior chamber of the eye because it cannot exit from the eye fast enough to keep up with aqueous production. As a consequence, the IOP increases. The increased IOP compresses the axons in the optic nerve and also may compromise the vascular supply to the optic nerve. This causes permanent damage.

The optic nerve carries vision from the eye to the brain. Some optic nerves seem more susceptible to IOP than others. While research is investigating ways to protect the nerve from elevated IOP, the only therapeutic approach currently available for glaucoma is to reduce the intraocular pressure.

The treatment of glaucoma is approached in a step-wise fashion. Medication often is the first treatment option. Administered either topically or orally, medications work to either reduce aqueous production or to increase outflow. Currently available medications have many serious side effects including congestive heart failure, respiratory distress, hypertension, depression, renal stones, aplastic anemia, sexual dysfunction, and death. Compliance with medication is a major problem; with estimates that over half of glaucoma doses are not taken.

When medication fails to adequately reduce the pressure in open angle glaucoma, laser trabeculoplasty often is performed. In laser trabeculoplasty, thermal energy from a laser is applied to a number of spots on the trabecular meshwork. It is believed that the laser energy alters the trabecular cells in some way. In approximately eighty percent of patients, aqueous outflow is enhanced and IOP decreases. However, the effect often is not long lasting and fifty percent of patients develop elevated pressure within five years.

Trabeculoplasty is not usually repeatable with beneficial effect on pressure. In addition, laser trabeculoplasty is not generally effective for young primary open angle glaucoma patients, nor is it effective for angle closure glaucoma and many secondary glaucomas.

If laser trabeculoplasty fails to sufficiently reduce the pressure, filtering surgery is generally performed. With filtering surgery, a hole is made in the sclera in the angle region. This hole allows aqueous to leave the eye through an alternate route. The most commonly performed filtering procedure is a trabeculectomy. In a trabeculectomy, an incision is made in the conjunctiva, the transparent tissue that covers the sclera. This exposes the sclera at the limbus. A partial thickness scleral flap is made and dissected approximately one-half thickness into the cornea. The anterior chamber is entered beneath the scleral flap and a section of deep sclera and trabecular meshwork is excised. An iridectomy, a hole in the thus exposed iris, is made. The scleral flap is loosely sewn back into place. The conjunctival incision is tightly closed. Post-operatively, the aqueous fluid passes through the hole, beneath the scleral flap, and collects in an elevated space beneath the conjunctiva called a filtration bleb. The fluid then is either absorbed through blood vessels in the conjunctiva or traverses across the conjunctiva into the tear film.

Trabeculectomy is associated with many problems, most of which are the result of the filtration bleb. Fibroblasts that are present in the episclera proliferate and migrate, and can scar down the scleral flap. This failure from scarring is particularly common in children, blacks, young adults, and in eyes which have had previous intraocular surgery. Of eyes that have an initially successful trabeculectomy, many will fail from scarring within three to five years after surgery. To minimize such scarring, surgeons now are applying antifibrotic agents such as mitomycin C (MMC) and 5-fluorouracil (5-FU) to the sclera and conjunctiva at the time of and after surgery. The use of these agents has increased the success rate of trabeculectomy, but also has increased the prevalence of hypotony and other serious complications such as intraocular infection. Hypotony is a problem that develops when aqueous flows out of the eye too fast, the eye pressure drops too low (usually less that 6.0 mmHg), and the structure of the eye collapses and vision decreases. Hypotony often requires a second trip to the operating room to tap suprachoroidal fluid, reform the anterior chamber, revise the sclerostomy, or repair bleb leakage.

Trabeculectomy creates a pathway for aqueous fluid to escape to the surface of the eye and into the blood stream. At the same time, it creates a pathway for bacteria that normally live on the surface of the eye and eyelids to enter the eye. If this happens, an internal eye infection, called endophthalmitis, can occur. Endophthalmitis can occur any time after trabeculectomy. The risk increases with the thin blebs that develop after the use of MMC and 5-FU. Another factor that contributes to infection is the placement of the bleb. Eyes that have trabeculectomy performed a the lower limbus have about five times the risk of eye infection that eyes that have a bleb superiorly, protected by the upper lid. Therefore, trabeculectomy is usually performed superiorly under the eyelid, in either the nasal of the temporal quadrant. In addition to scarring, hypotony, and infection, there are other complications of trabeculectomy. The bleb can tear and leak causing hypotony. It can also disrupt the normal tear film, leading to blurred vision and discomfort. Patients with blebs generally cannot safely wear contact lenses. The overwhelming majority of the complications from trabeculectomy stem from the fact that fluid is being diverted from inside the eye to the external surface of the eye resulting in an elevated filtration bleb.

When trabeculectomy does not successfully lower the eye pressure, the next surgical step is often an aqueous shunt device. An aqueous shunt device of the prior art is a silicone tube that is attached at one end to a plastic (polypropylene or other synthetic material) plate. With an aqueous shunt device, an incision is made in the conjunctiva and Tenons, exposing the sclera. The plastic plate is sewn to the surface of the eye posteriorly, usually over the equator of the eye between two rectus muscles. A full thickness hole is made into the eye at the limbus, usually with a needle of approximately 22 gauge. The tube, which is connected to the plate, is inserted into the eye through this hole. The external portion of the tube is covered with either donor sclera or preserved pericardium. The conjunctival and Tenons incisions are closed tightly. Many problems exist with the current technology of aqueous shunt devices including scarring, failure, hypotony, corneal decompensation, tube erosion, suprachoroidal effusion and/or hemorrhage, and infection.

With prior art aqueous diversion devices, aqueous drains out of the eye through the silicone tube to the surface of the eye at the location of the plate or reservoir. Deeper orbital tissues then absorb the fluid. The outside end of the tube is protected from fibroblasts and scarring by the plastic plate. Many complications are associated with aqueous shunt devices. A thickened wall of scar tissue that develops around the plastic plate offers some resistance to outflow and in many eyes limits the reduction in eye pressure. In some eyes, hypotony develops because the flow through the tube is not restricted. Some physicians tie an absorbable suture around the tube and wait for the suture to dissolve post-operatively, at which time enough scar tissue, the surgeon hopes, have formed around the plate. Some devices contain a pressure-sensitive valve within the tube, although these valves may not function properly and may limit the IOP lowering which is necessary for severely damaged and vulnerable optic nerves. The surgery involves operating in the posterior orbit and many patients develop an eye muscle imbalance and double vision. With prior art aqueous shunt devices, a pathway is created for bacteria to get into the eye and endophthalmitis can occur.

Most of the problems with current glaucoma treatment devices and procedures occur because aqueous is drained from the inside of the eye to the surface of the eye. A need exists, then for a more physiologic system to enhance the drainage of aqueous from the anterior chamber to the episcleral venous system. In the vast majority of glaucoma patients, the resistance problem lies between the anterior chamber and Schlemm's canal. The canal itself, the collecting channels and the episcleral venous system are all intact. Enhancing aqueous flow directly into Schlemm's canal would eliminate most of the complications encountered with filtration surgery because a bleb is avoided. This would be physiologic because the canal is part of the normal outflow system and is biologically engineered to handle the normal volume of aqueous.

Recently, tubes have been developed shunting aqueous directly from the anterior chamber to Schlemm's canal. Unfortunately, although these tubes have resulted in lowering of IOP, the pressures are not low enough (mid to low teens and single digit pressures) for severely damaged and vulnerable optic nerves. Apparently some additional resistance to aqueous outflow exists between Schlemm's canal and the aqueous veins.

A need, therefore, exists for shunting aqueous directly from the anterior chamber past the resistance (in the trabecular meshwork and between Schlemm's canal and the aqueous veins). This could be accomplished with tubes between the anterior chamber and aqueous veins, collector channels, and/or veins (even if extraorbital). This would require exquisitely fine tubes, appropriate for the small size of the aqueous veins, collector channels, and veins. Because of the small size it may be necessary to cannulate multiple channels/vessels to accommodate sufficient drainage of aqueous. Aspects of the invention include methods for attachment, such as with sutures, to the scleral surface. Various innovations and novel instrumentation would also be needed.

### II. SUMMARY OF THE INVENTION

In accordance with one aspect of the present invention, a method for draining aqueous includes creating a limbal incision and inserting a drainage tube between an associated anterior chamber and at least one of the group comprising: at least one aqueous vein, at least one collector channel, at least one vein, and at least one distal vein.

In accordance with another aspect of the present invention, the limbal incision is tunneled from a posterior location in the sclera.

In accordance with another aspect of the present invention, the limbal incision is a substantially straight hole through the limbus into the anterior chamber.

In accordance with another aspect of the present invention, the tube is located on the sclera.

In accordance with another aspect of the present invention, the method includes covering the tube with graft material.

In accordance with another aspect of the present invention, the method includes covering the tube with the scleral flap.

In accordance with another aspect of the present invention, the method includes inserting the drainage tube between the anterior chamber and at least one collector channel.

In accordance with another aspect of the present invention, the method includes inserting the drainage tube between the anterior chamber and at least one distal vein.

In accordance with another aspect of the present invention, the method includes inserting the drainage tube into the anterior chamber with micro forceps.

In accordance with another aspect of the present invention, the method includes suturing the tube in place via fixation plates and suture holes.

In accordance with another aspect of the present invention, the method includes inserting multiple drainage tubes to multiple aqueous veins, collector channels, or systemic veins.

In accordance with another aspect of the present invention, the method includes measuring episcleral venous pressure to select optimal site for tube placement.

In accordance with another aspect of the present invention, the episcleral venous pressure is measured using a micromanometer.

In accordance with another aspect of the present invention, a method for draining aqueous includes creating an incision in the sclera, opening a scleral flap, and inserting a drainage tube between an associated anterior chamber and at least one distal vein.

In accordance with another aspect of the present invention, the drainage tube bypasses the aqueous veins and collector channels.

In accordance with another aspect of the present invention, the method includes utilizing a lens to magnify the incision.

In accordance with another aspect of the present invention, the method includes inserting a pump to ensure that intraocular pressure is maintained.

In accordance with another aspect of the present invention, the method includes adjusting the speed of the pump to maintain intraocular pressure.

In accordance with another aspect of the present invention, an apparatus for draining aqueous includes a tube, the tube having a proximal and a distal end and a flow opening and at least one retaining device, for holding the tube in place.

In accordance with another aspect of the present invention, the retaining device includes at least one fixation plate and at least one suture hole.

In accordance with another aspect of the present invention, the retaining device includes a nipple, the nipple attached to the proximal end.

In accordance with another aspect of the present invention, the apparatus includes a one-way valve.

In accordance with another aspect of the present invention, the apparatus includes a tapered tip and the nipple being angled at substantially a 135 degree angle with respect to the flow opening.

In accordance with another aspect of the present invention, the apparatus includes multiple tubes, the multiple tubes located at the distal end.

In accordance with another aspect of the present invention, the apparatus includes a micromanometer.

In accordance with another aspect of the present invention, the micromanometer includes a transducer, a bulb, a wire connecting the transducer to the bulb, an air intake for the bulb, and a pressure sensor.

In accordance with another aspect of the present invention, the apparatus includes a tube extender, the tube extender having serrated ridges.

In accordance with another aspect of the present invention, the apparatus includes a chuck, the chuck includes a lens, a port, and a channel.

In accordance with another aspect of the present invention, the apparatus includes connectors, the connectors having a shape chosen from the group comprising T-shaped and Y-shaped.

In accordance with another aspect of the present invention, the connectors have serrated ridges.

In accordance with another aspect of the present invention, the apparatus includes a cowl, the cowl covering the tube, and at least one fixation hole for holding the cowl in place.

In accordance with another aspect of the present invention, the apparatus includes a pump mechanism, the pump attached the drainage tube.

In accordance with another aspect of the present invention, the apparatus includes a feedback device, the device connected to the pump.

In accordance with another aspect of the present invention, an apparatus for draining aqueous includes a tube, the tube having a proximal and a distal end and a flow opening and multiple insertion tubes located at the distal end.

In accordance with another aspect of the present invention, the pump mechanism is adapted to draw the aqueous humor from the anterior chamber through the tube and into one of the group comprising: aqueous vein, collector channel, vein, and angular vein.

In accordance with another aspect of the present invention, the pump mechanism further comprises a power source operatively connected thereto.

In accordance with another aspect of the present invention, the pump mechanism has length, width, and thickness dimensions of approximately 2 mm by approximately 2 mm by approximately 500 microns, respectively.

In accordance with another aspect of the present invention, the pump mechanism is implanted posterior to an associated limbus.

In accordance with another aspect of the present invention, the pump mechanism further comprises a posterior surface, the posterior surface being concave.

In accordance with another aspect of the present invention, the pump mechanism further comprises a power source having a posterior surface, the posterior surface being concave.

In accordance with another aspect of the present invention, the pump mechanism has length, width, and thickness dimensions of approximately 6 mm, approximately 10 mm, and approximately 3 mm, respectively.

In accordance with another aspect of the present invention, the pump mechanism is adapted to operate on a demand basis, such that the required flow through the tube to achieve desired intraocular pressure varies according to diurnal fluctuation in aqueous production.

In accordance with another aspect of the present invention, the apparatus includes a feedback mechanism for monitoring work performed by the pump mechanism to achieve the desired intraocular pressure.

In accordance with another aspect of the present invention, the pump mechanism is adapted to be adjusted without having to surgically dissect tissues to expose a large portion of the pumping mechanism.

In accordance with another aspect of the present invention, the apparatus includes pressure reading means for transmitting intraocular pressure readings to a controller.

In accordance with another aspect of the present invention, the pump mechanism comprises a wafer.

In accordance with another aspect of the present invention, the wafer is surrounded by a microchip and an insulating protective layer.

In accordance with another aspect of the present invention, the pump mechanism is located on the surface of an associated eye.

### III. BRIEF DESCRIPTION OF THE DRAWINGS

The invention may take physical form in certain parts and arrangement of parts, at least one embodiment of which will be described in detail in this specification and illustrated in the accompanying drawings which form a part hereof and wherein:

FIG. 1 is a front view and a cross-sectional view of the tube;

FIG. 1A is a front and cross-sectional view of the device showing a nipple at the proximal end to prevent retraction from the anterior chamber;

FIG. 1B is a front and cross-sectional view of the device showing fixation daggers;

FIG. 1C is a front and cross-sectional view of the device showing fixation holes for suture fixation;

FIG. 1D is a front and cross-sectional view of the device showing angulation of the nipple to lie flush against the peripheral cornea and trabecular meshwork;

FIG. 1E is a cross-sectional view of the device showing a valve to allow flow only in the desired direction of aqueous drainage;

FIG. 1F is a front and cross-sectional view of the device showing a tapered tip.

FIG. 1G is a front and cross-sectional view of the device showing different calibers (inner and outer) at different positions on the tube;

FIG. 1H is a front and cross-sectional view of the device showing a sharp tip (such as a beveled tip) for insertion into venous structure;

FIG. 1I is a front and cross-sectional view of the device showing fixation tabs/holes for suture fixation at different locations depending upon the anticipated site of implantation;

FIG. 1J is a front and cross-sectional view of another embodiment of FIG. 1I;

FIG. 1K is a front and cross-sectional view of the device showing tubes for implantation into multiple collector channels and/or veins;

FIG. 1L is a perspective view of the anterior chamber of the eye, showing the tube, as shown in FIG. 1K, inserted into the collector channel;

FIG. 2 is a front and cross-sectional view of the device showing movable fixation plates for suture fixation of tube or extender;

FIG. 3 is a cross-sectional view showing micro forceps with a channel to insert the tube with/without nipple;

FIG. 4 is a perspective view of the anterior chamber of the eye, showing retrograde injection of dye into aqueous veins to identify ostia of collector channels in Schlemm's canal;

FIG. 5A is a front and cross-sectional view of a micromanometer to measure episcleral venous pressure (EVP);

FIG. 5B is another embodiment of the device shown in FIG. 5A;

FIGS. 6A and 6B are front and cross-sectional views of hollow needles for passage of tubes through tissue including outside the orbit to the vicinity of the angular vein (straight and curved);

FIG. 7 is a front, top, and cross-sectional view of a chuck, lens, and channel;

FIG. 8 is a front and cross-sectional view of a tube extender with serrated ridges to maintain traction on tube;

FIG. 9A is a top view a cowl to protect tube, showing fixation tabs or holes;

FIG. 9B is a front view of FIG. 9A;

FIG. 10 is a front and cross-sectional view of an injection cannula;

FIG. 11A is a front and perspective view of a “T” connector;

FIG. 11B is a front and perspective view of a “Y” connector;

FIG. 12 is a front and perspective view of the connectors of FIG. 11 with serrated ridges to maintain friction between the connector and tube;

FIG. 13 is a perspective view of the anterior chamber and a fluorescein injection;

FIG. 14 is a front and perspective view of an injector;

FIG. 15A is a perspective view of the anterior chamber showing tube placement into an aqueous vein, into a collector channel, and into a vein;

FIG. 15B is gonioscopic view of an insertion of the tube;

FIG. 16 is a perspective view of the anterior chamber showing tube placement into aqueous vein ostia—collector channel, into aqueous vein ostia—Vein (surface of eye or orbital), and into aqueous vein ostia—Distant vein such as angular or other similar vein;

FIG. 17A shows a cross-sectional view of a tube on the surface of sclera;

FIG. 17B shows a cross-sectional view of a tube covered by pericardium, sclera, or other similar graft material;

FIG. 17C shows a cross-sectional view of a tube embedded under partial—thickness sclera flap;

FIG. 18 is cross-sectional view of a human eye, showing the retinal pigmented epithelium, the sclera, the choroids, the retina, the fovea, the macula, the optic nerve, the vitreous humor, the conjunctiva, the cornea, the iris, the pupil, the lens, the aqueous, and the trabecular meshwork/schlemm's canal;

FIG. 19 is a perspective view of the pump mechanism on the tube;

FIG. 20 is a perspective view of the pump mechanism; and,

FIG. 21 is a perspective view of the pump mechanism showing the channels.

### IV. DESCRIPTION OF THE INVENTION

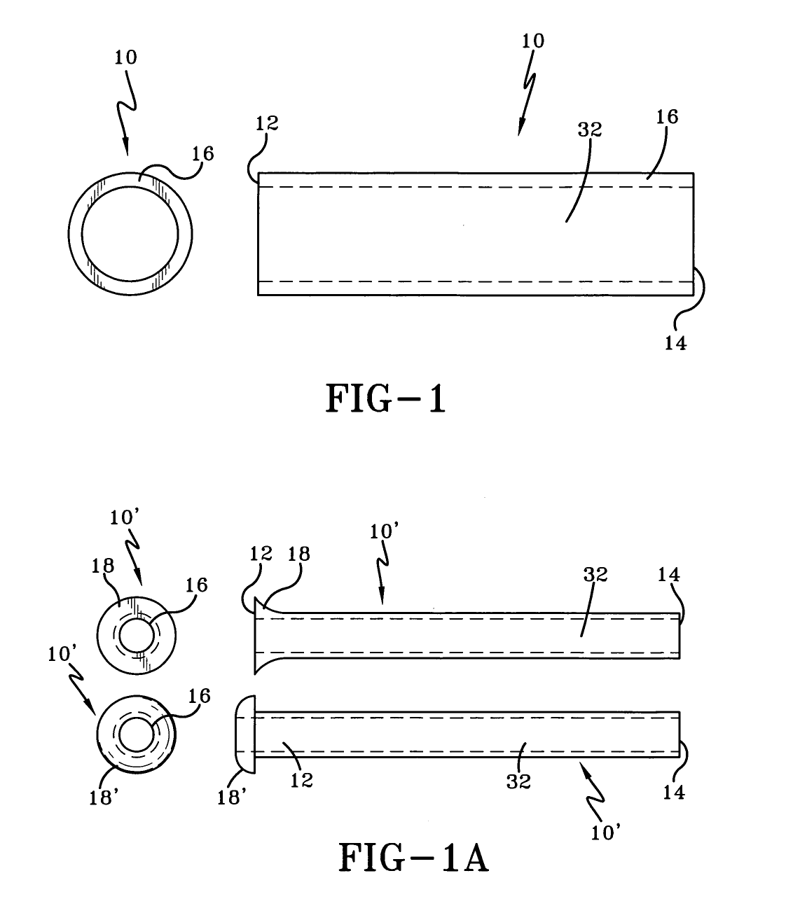

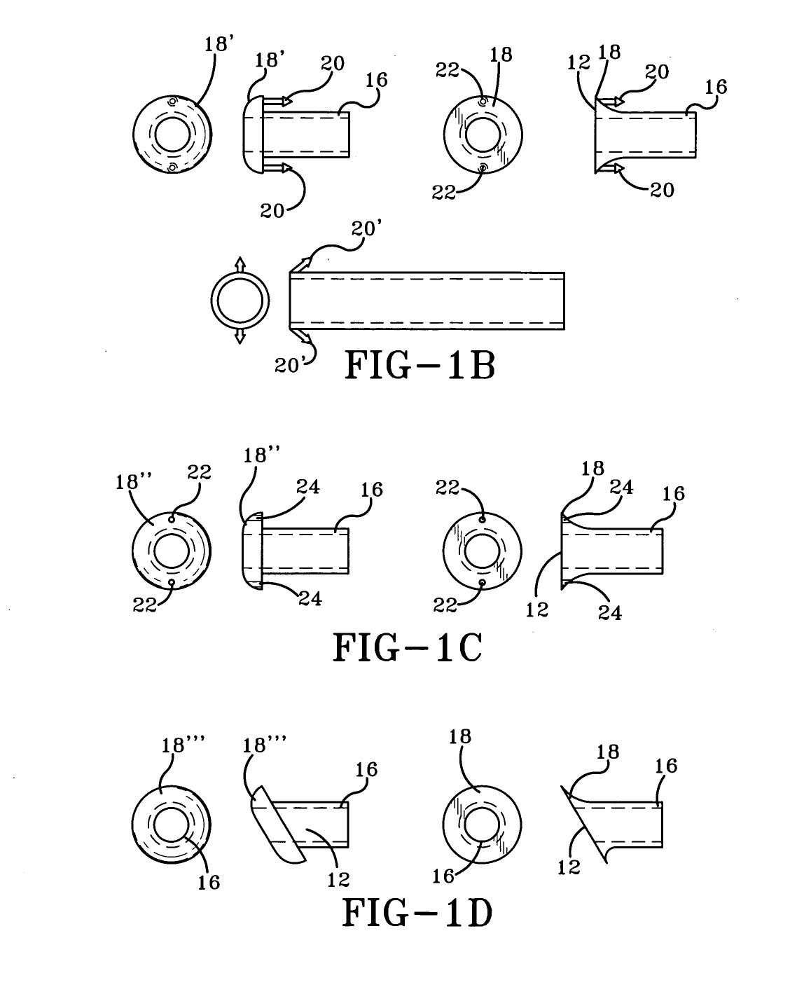

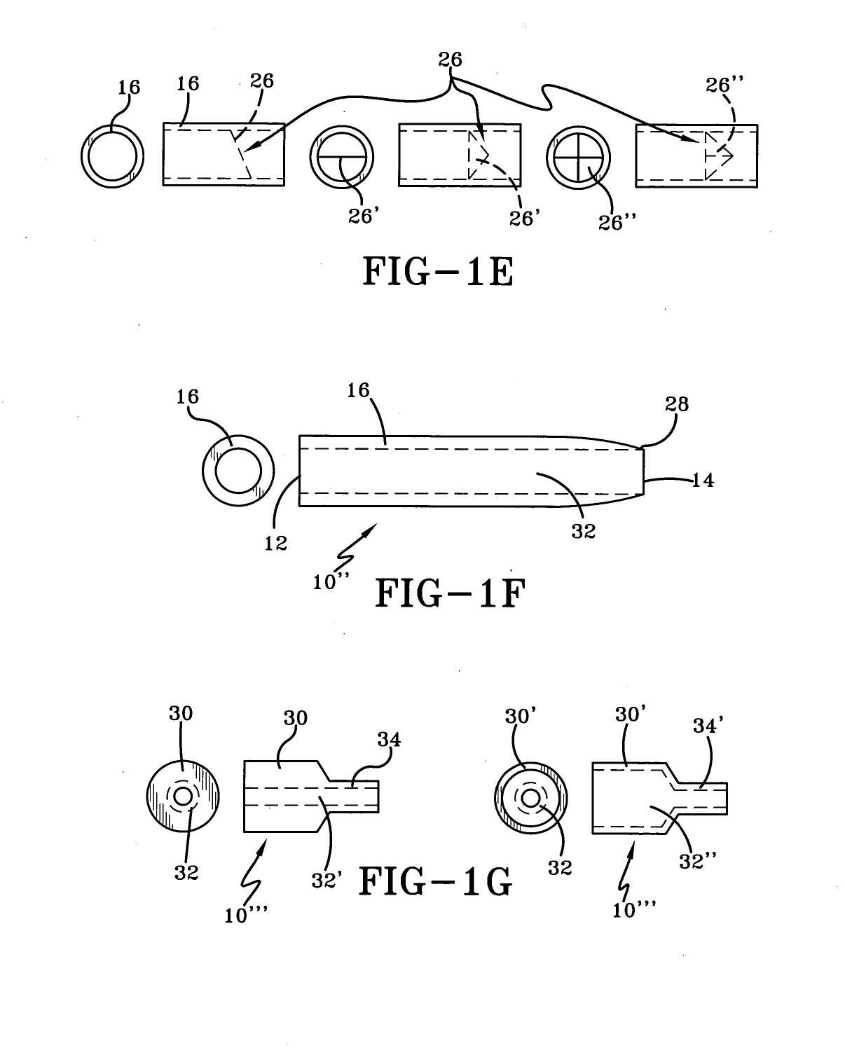

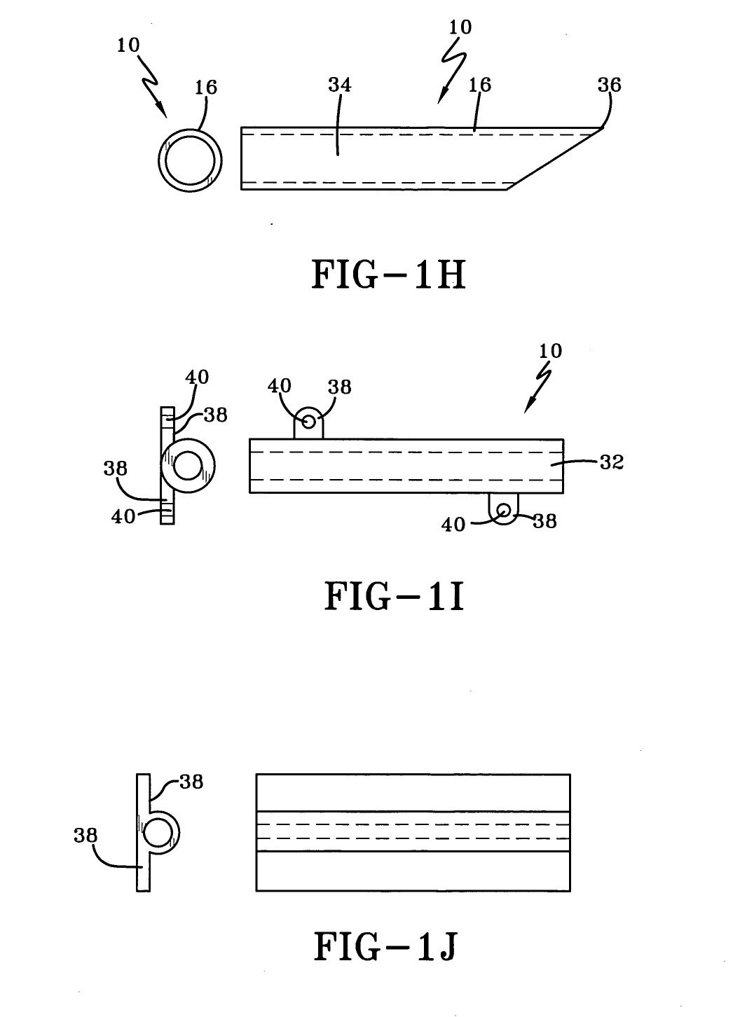

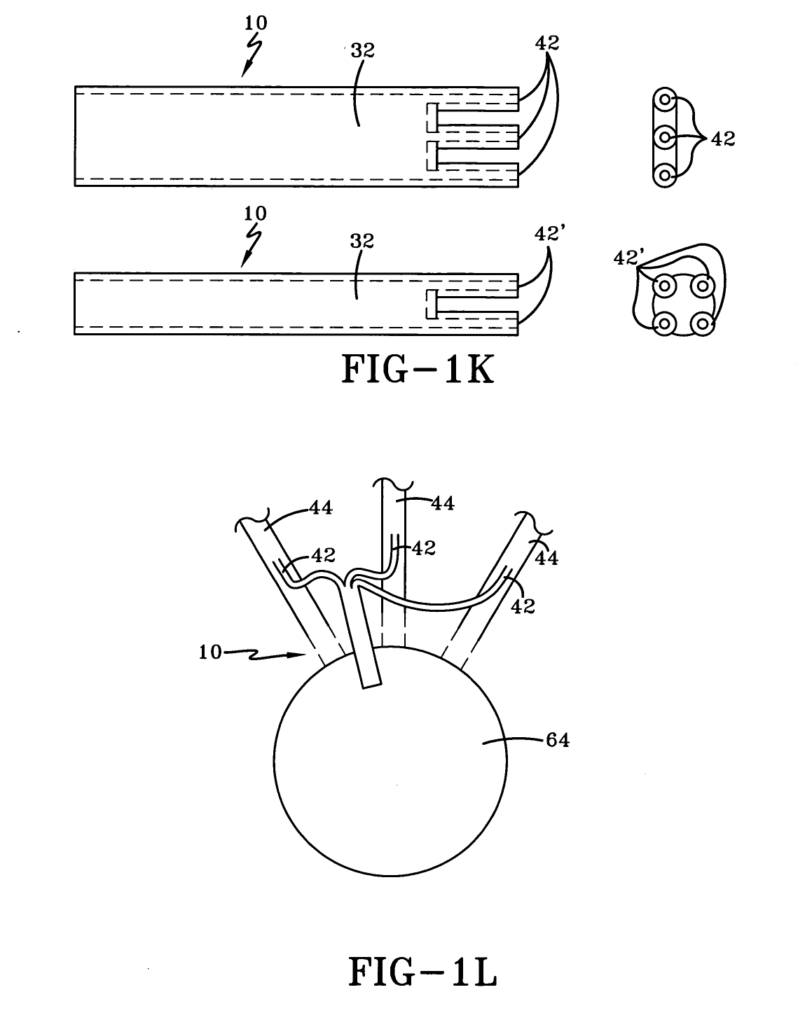

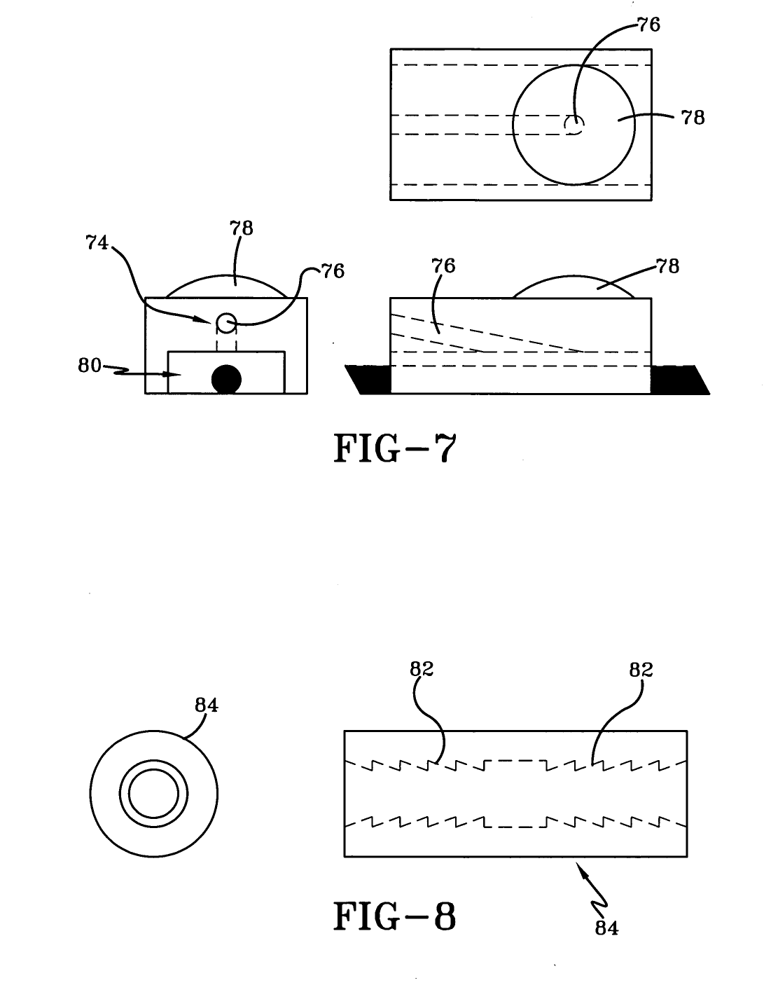

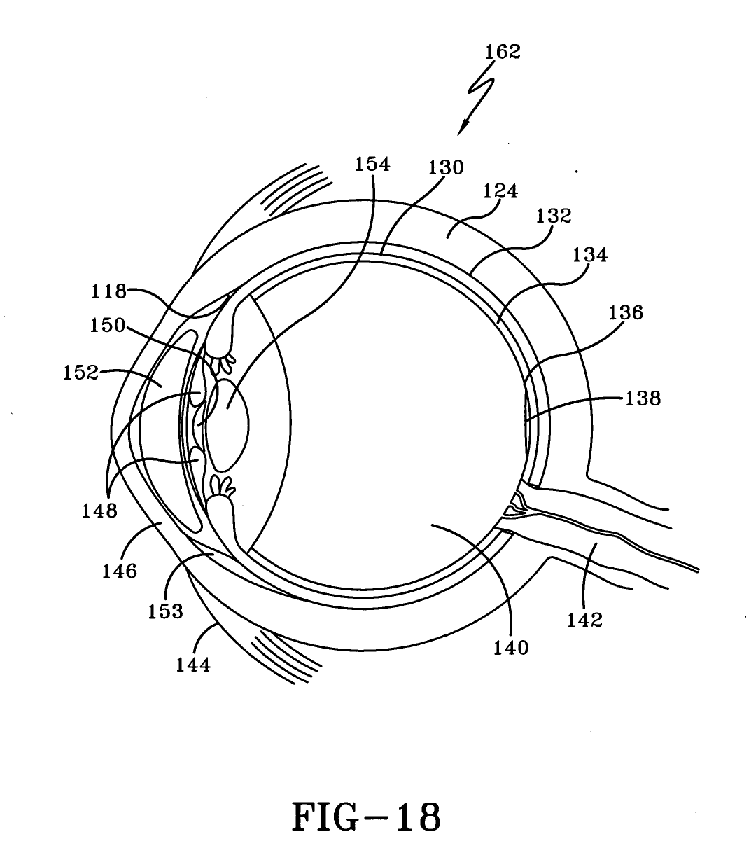

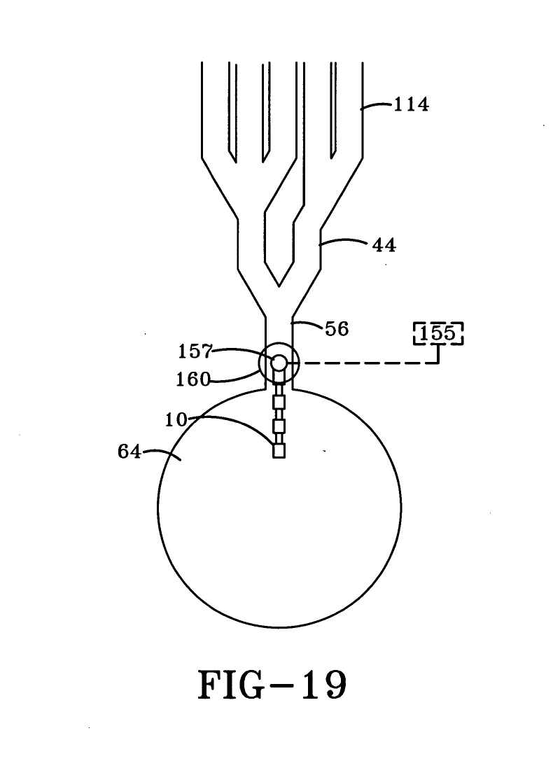

Referring now to FIGS. 1-21 wherein the showings are for purposes of illustrating at least one embodiment of the invention only and not for purposes of limiting the same, FIG. 18 shows trabecular meshwork/Schlemm's canal 118, sclera 124, retinal pigmented epithelium 130, choroids 132, retina 134, fovea 136, macula 138, vitreous humor 140, optic nerve 142, conjunctiva 144, cornea 146, iris 148, pupil 150, aqueous 152, limbus 153, and lens 154.

FIGS. 1-1L show various embodiments of the tube 10. The tube 10 has proximal 12 and distal 14 ends, a tube wall 16, and flow opening 32. In one embodiment of the invention, the tube 10′ has a nipple 18 at the proximal end 12 to prevent retraction from the anterior chamber 64. The nipple 18 also eliminates the need for a lengthy tube extending into the anterior chamber 64. The shape and angle of the nipple 18, 18′, 18″, 18′″ can vary, and can include fixation holes 24 for suture fixation. The nipples 18, 18′, 18″, 18′″ can also be angled as shown in FIG. 1D. The angled nipple 18′″ lies flush against the peripheral cornea 146 and trabecular meshwork 118. The nipple 18, 18′, 18″, 18′″ can also have devices for securing the tube 10 in place, such as fixation daggers 20, which can be attached at dagger attachments 22. It is to be understood that the nipples and fixation daggers are merely one embodiment of the invention, and any retaining device that ensures that the tube remains in place can be used, as long as chosen using sound engineering and medical judgment.

With reference now to FIG. 1E, the tube 10 can have a valve 26, 26′ 26″ inserted into the flow opening 32. The valve 26 is designed to allow flow only of aqueous 152 only in the desired direction of aqueous drainage. This control of flow will protect the eye from elevated IOP due to elevated venous pressure during valsalva and other conditions leading to elevated episcleral venous pressure. The valve 26 would also prevent reflux of blood into the anterior chamber 64 during the above situations. The configuration and operation of the valve 26, 26′, 26″ can be of any kind, as long as chosen using sound engineering and medical judgment, and as long as the valve 26, 26′, 26″ is capable of controlling the flow of aqueous 152.

With reference now to FIGS. 1F-1H, the tube 10, 10″, 10′″ may have many different shapes. For example, the tube 10″ may have a tapered tip 28 at distal end 14 to allow easier insertion. The tube 10′″ may have different calibers at different positions on the tube 10′″. The tube 10′″ may have a tapered wall 30 and a narrow wall 34, which can have either flow opening 32′ or flow opening 32″, as shown in FIG. 1G. FIG. 1H shows the tube 10 with a beveled tip 36, which allows for easier insertion into the venous structures. It is to be understood that the present invention is not limited by the various embodiments of tube 10, 10′, 10″, 10′″.

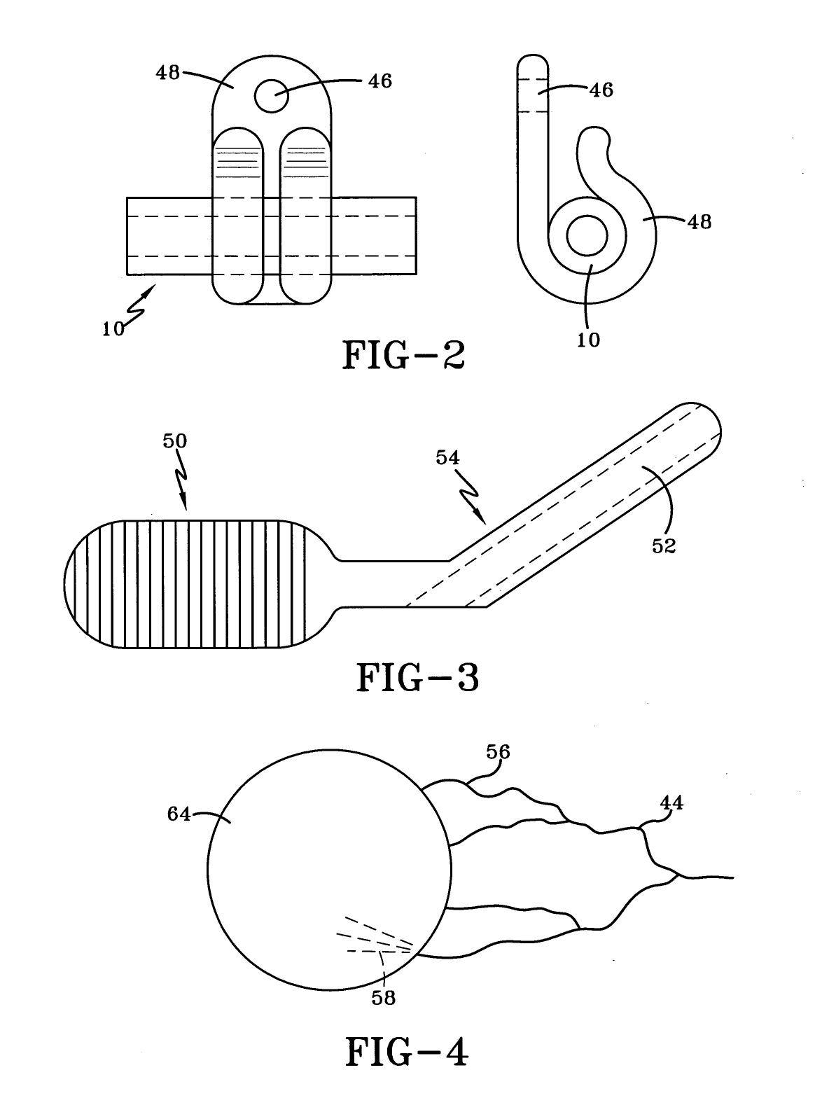

With reference now to FIGS. 1I and 1J, the tube 10 is secured in place by fixation plates 38 and suture holes 40. The number, shape, and placement of the fixation plates 38 and suture holes 40 are not limited by this invention, and can be any number, shape, and placement, as long as chosen using sound engineering and medical judgment. The plates 38 can be made of the same material as the tube 10 or a different material. In this embodiment, the tube is secured to the sclera 124. FIG. 2 shows movable fixation plate 48 and suture hole 46 for attaching the tube 10 to the sclera 124. The plate 48 can be adjusted along the length of the tube 10 as necessary for proper placement. The plate 48 can be a flexible tube that substantially encircles the tube 10.

With reference now to FIG. 1K, the tube 10 can have multiple insertion tubes 42 at the distal end 14. These tubes 42 can be any number desired, and any configuration desired, as long as chosen using sound engineering and medical judgment. The tubes 42 allow for insertion into multiple venous structures at the same time. The tubes 42 are sufficiently flexible, and of sufficient length to be inserted into the aqueous veins 56, collector channels 44, or other distal veins 114. FIG. 1L shows the tubes 42 connected between the anterior chamber 64 and multiple collector channels 44.

With reference now to FIG. 3 micro forceps 50 are shown with a lumen 52, and angled portion 54 for inserting the tube 10 into the eye. The tube 10 is placed in the lumen 52, and then the forceps 50 are used to insert the tube 10 into the eye. As shown in FIGS. 6A and 6B, the lumen 52, 52′ can be straight, or curved up to approximately a 270 degree arc. FIG. 14 shows an inserter 108 that helps insert the tube 10. The inserter 108 has a proximal end 110 and a tip 112. The tip 112 is inserted in then, when the proximal end 110 is removed, the tube 10 stays in place.

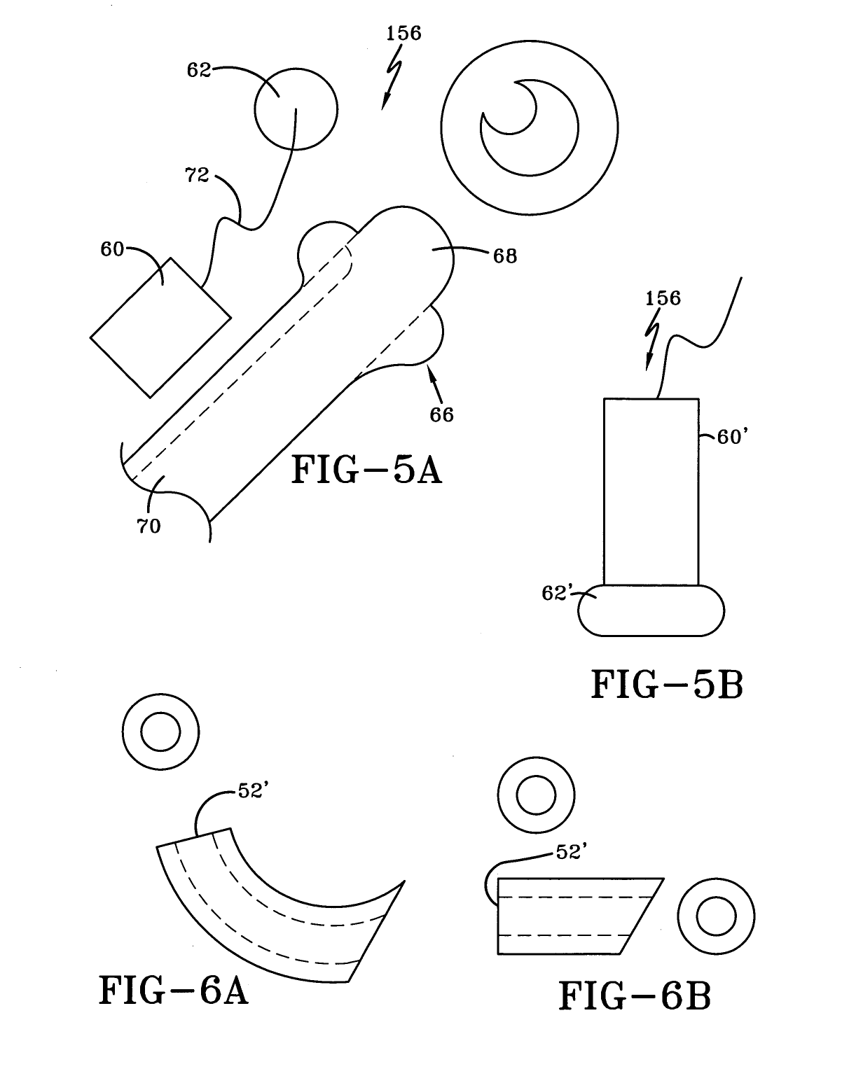

With reference now to FIGS. 4-7, prior to inserting the tube 10, in this embodiment, a retrograde injection of dye 58 into the aqueous veins 56 identifies the ostia of the collector channels 44 into Schlemm's canal. A micromanometer 156 is used to measure episcleral venous pressure to rule out malformations to select an optimal site for tube placement. The micromanometer 156 has a transducer 60, a wire 72, and bulb 62. The bulb 62 has an air intake 70, bulb sides 66, and a pressure sensor 68. The transducer 60, which may be connected to the bulb 62 or remotely connected via wire 72, measures/displays the bulb pressure. The inflation and compression of the bulb 62 measures the episcleral venous pressure. During insertion of the tube 10, a chuck 74 is used for magnification for visualization, irrigation, diamond, or other microknives, and insertion. The chuck 74 has a lens 78, port 76 for the blade, and channel 80 for the tube 10. The lens 78 minimizes or eliminates the need for additional magnification by a conventional microscope.

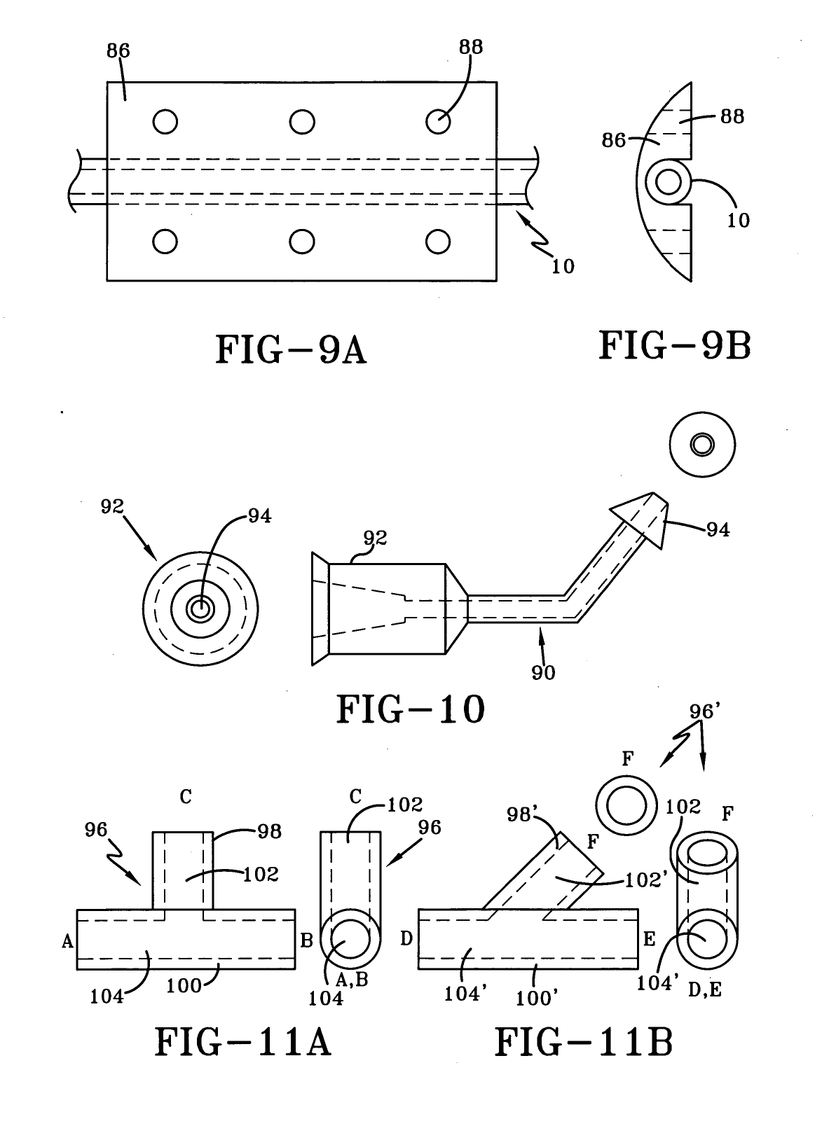

With reference now to FIG. 8, once the tube 10 has been inserted, a cylindrical tube extender 84 with serrated ridges 82 can be used to maintain traction on the tube 10. In FIG. 9, a rigid cowl 86, with fixation holes 88 is shown. The cowl 86 helps protect the tube 10 once it has been inserted. The holes 88 help hold the cowl 86 in place on the sclera 124.

With reference now to FIG. 10, an injection cannula 90 is inserted into the proximal end 12 of the tube 10 to flush out the tube 10. The cannula 90 has a tip 94 and an adapter 92. The tip 94 is inserted into the opening of the tube 10 and fluid is passed through the adapter 92 into the tube 10.

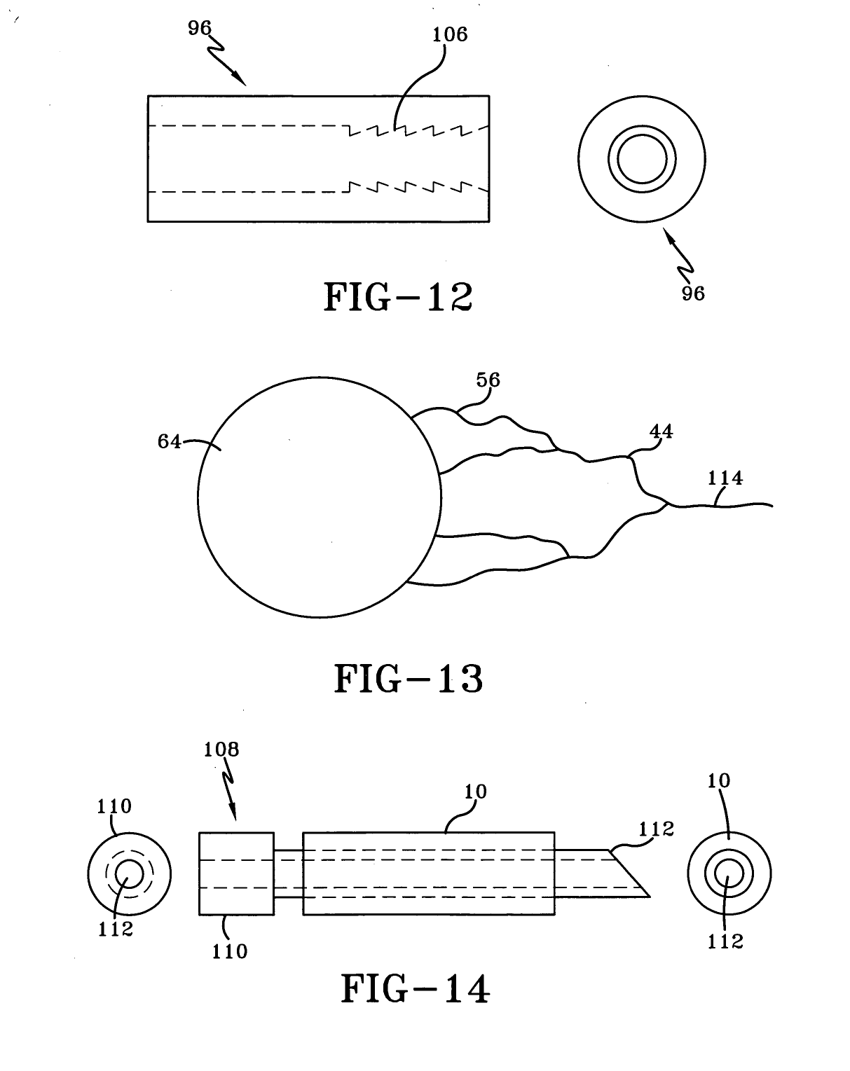

With reference now to FIGS. 11A-12, “T” or “Y” connectors 96, 96′ serve as safety valves for conventional setons and can also salvage a failing seton operation. The connectors 96, 96′ can have serrated ridges 106 to maintain friction between the connector 96, 96′ and the tube 10. The connectors 96, 96′ have specific shapes according the location and shape of the tube 10. The connectors 96, 96′ have a stem portion 98, 98′, a T portion 100, 100′, a flow portion 102, 102′, and a flow portion 104, 104′. The serrated ridges 106 can be located at any of the openings of the connectors 96, 96′.

With reference now to FIG. 13, AC fluorescein angiography is used to identify aqueous outflow anatomy. Fluorescein is injected intravenously or via corneal paracentesis into the anterior chamber 64. Fluorescein is visible with cobalt blue light in aqueous veins 56, collector channels 44, and veins 114.

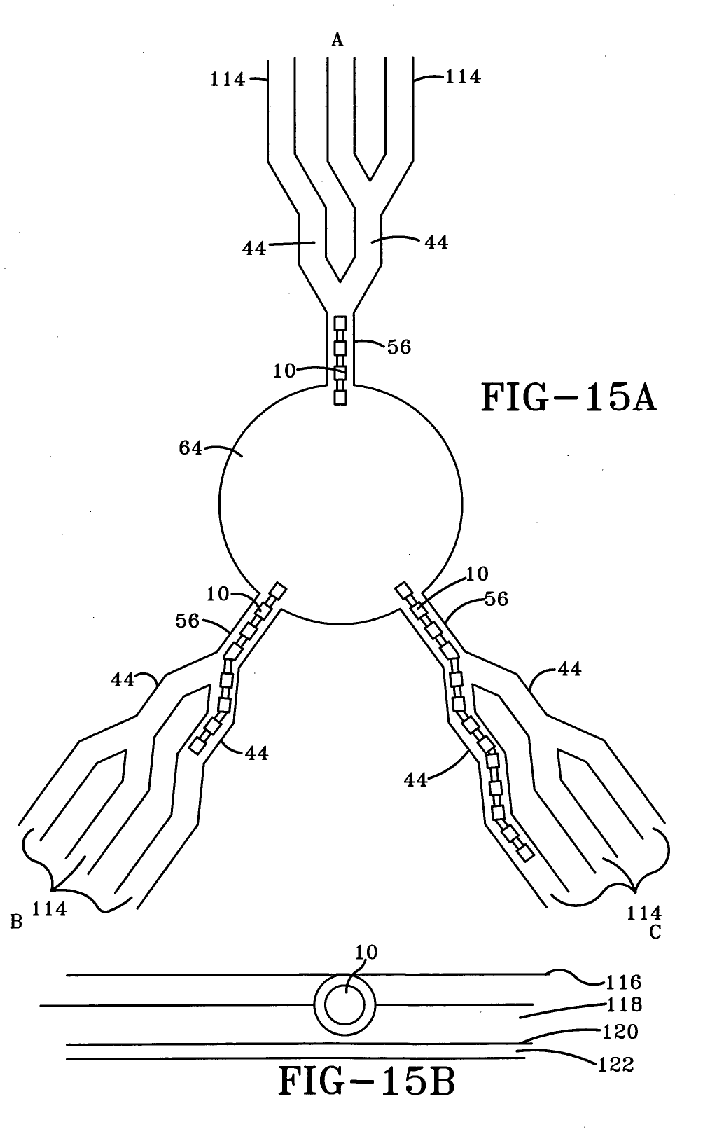

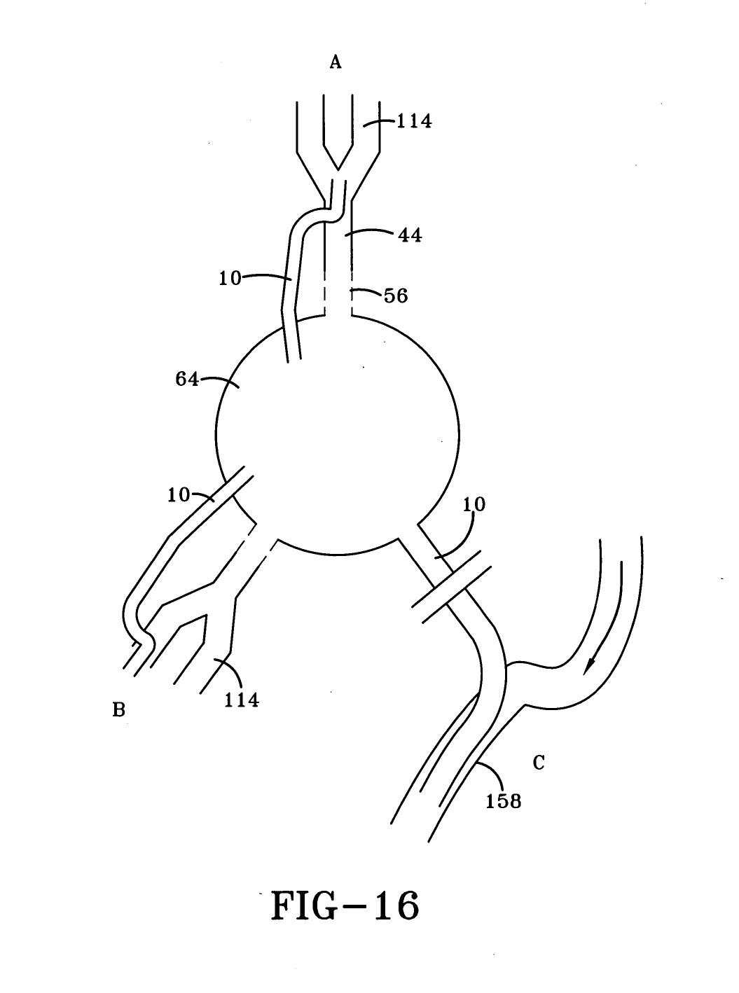

With reference now to FIGS. 15A-17, various insertion pathways are shown. In FIG. 15A, the tube 10 is shown inserted into the anterior chamber 64, and inserted into the aqueous veins 56, the collector channels 44, and the veins 114. It is to be understood that, as shown in FIGS. 15 and 16, the tube 10 can be in any or all of these. The tube 10, as shown in FIG. 16, can bypass the aqueous vein 56 and be inserted into the collector channel 44, or can bypass both the aqueous vein 56 and the collector channel 44, and be inserted into the vein 114. In another embodiment, the tube 10 could bypass the aqueous vein 56, the collector channel 44, and the vein 114, and be inserted into a distant vein 158, such as the angular vein 158.

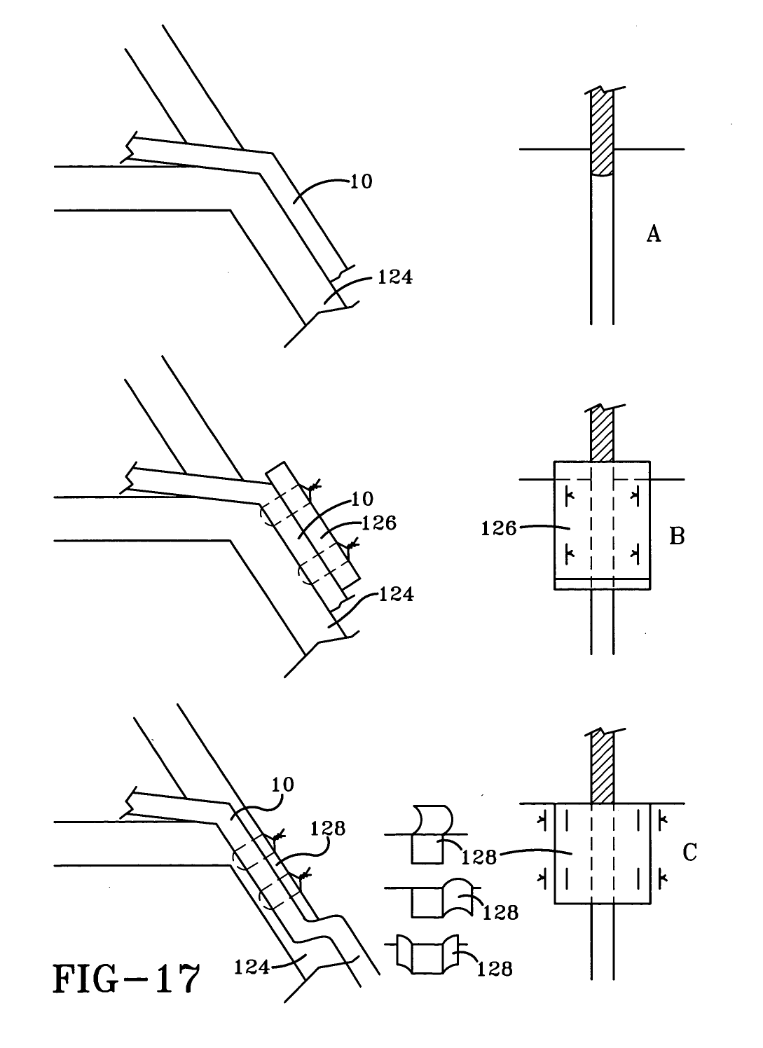

With reference now to FIG. 17, in several embodiments, the tube 10 can be 1) on the surface of the sclera 124, 2) covered by pericardium 126, sclera 124, or other similar graft material 126 (The graft material 126, in this embodiment, is sutured to the sclera 124 to cover the tube 10, and hold it in place), or 3) embedded under a partial thickness of the scleral flap 128.

With reference now to FIGS. 19-21, the pumping mechanism 160 has a posterior surface, which is substantially concave in order to conform to the surface of the eye 162, when the pumping mechanism 160 is between the rectus muscles.

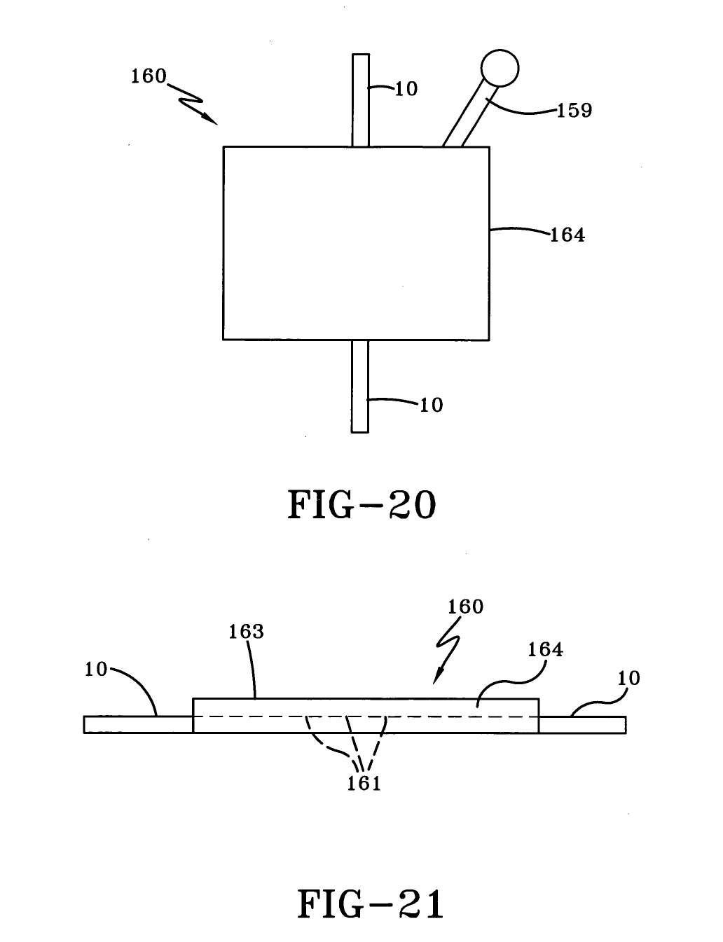

In this embodiment, as shown in FIGS. 19-21, the pumping mechanism 160 is utilized in conjunction with the tube 10 in order to provide for better outflow of aqueous humor 152. The pumping mechanism 160 is adapted to draw aqueous humor from the anterior chamber 64 and direct the aqueous humor into the tube 10 and into the aqueous vein 56, collector channel 44, vein 114, or distant vein 158. The pumping mechanism 160 may take the form of a nanopump, which is generally a wafer 164 with tiny channels 161, which move polar solution by electric current, as shown in FIGS. 19-21. Such pumps 160 are available through iMEDD, Inc., which has its principal place of business at 1224 Kinnear Road, Suite 130, Columbus, Ohio 43212. It is to be understood, however, that any pumping mechanisms that meets the size and flow requirements could be used. The pump 160 is encased in an insulative material 163, which protects against electrical surges. The material 163 also serves to protect the pump 160 from damage by force exerted by the eyelid during blinking or through dabbing or rubbing of the eye 162. The pump 160 can be made of any material chosen using sound engineering and medical judgment. The insulative material 163, in this embodiment is silicone, but can be any biologically inert material. In one embodiment of the present invention, the nanopump channels 161 should have a minimum cross-sectional dimension between 2 and 100 nanometers and, preferably, between 10 and 30 nanometers. The channels 161 are shown as the spaces in the dotted line, shown as the wafer 164 in FIGS. 20 and 21.

It is also desirable for the nanopump 160 to have a voltage potential of approximately 0.5 to approximately 20 volts. Such a nanopump 160 is disclosed in a U.S. patent application, filed on Jun. 15, 2001, entitled Nanopump Apparatus and Method, co-invented by Derek Hansford, Ph.D., Assistant Professor of The Ohio State University and Rob J. Walczak, a scientist at iMEDD, Inc.

FIGS. 19-21 illustrate one embodiment of the pumping mechanism 160 in operative association with the tube 10. Turning to FIG. 20, the wafer 164 may range from approximately two to four millimeters in length and approximately two to four millimeters in width. Wires 159 extend from the wafer 164 to a power source 157, which may be batteries. The thickness of the working portion of the pumping mechanism 160 may be as thin as approximately three microns, and the total thickness of the wafer 164 may be as thin as approximately 200 microns.

In this embodiment, the power source 157 would be positioned between rectus muscles 50 (although it is to be understood that any location may be chosen for the power source 157, as long as chosen using sound engineering and medical judgment. If wires 159 are utilized between the power source 157, the tube 10, and pumping mechanism 160, the wires 159 should be insulated. In this embodiment, power source 157 may be up to approximately thirteen millimeters in diameter and up to approximately three millimeters in thickness. In this embodiment, the dimensions of the pumping mechanism 160 should be fairly small. For example, length, width, and thickness dimensions may be about 2 mm by about 2 mm by approximately 500 microns, respectively. A posterior surface of the power source 157 may be substantially concave in order to conform to the surface of the eye 162.

In another embodiment of the invention, the pumping mechanism 160 and the power source 157 could be much larger, such as approximately 6 mm by approximately 10 mm by approximately 3 mm in length, width, and thickness, respectively.

In another embodiment of this invention, the pumping mechanism 160 has a filter (not shown) to protect the pumping mechanism 160. The pumping mechanism 160 could also be treated with heparin, or other agents, to avoid clogging. In one embodiment, the operation of the pumping mechanism 160 would be utilized on a demand basis, such that the required flow through the tube 10 to achieve the desired intraocular pressure would vary according to the diurnal fluctuation in aqueous production. A feedback mechanism 155 communicates with the pumping mechanism 160 to achieve the required flow rate, which results in the desired intraocular pressure. The feedback mechanism 155 may be integral with the pumping mechanism 160 or it may be a stand-alone unit. In order to avoid excessively low intraocular pressure, the pumping mechanism 160 may operate slower to decrease output of aqueous humor.

It is contemplated to be within the scope of the present invention that the pump speed be adjustable without having to surgically dissect tissues to expose a large portion of the pumping mechanism 160. Telemetry may also be utilized to provide intraocular eye pressure readings without the necessity of examining the patient.

It is to be understood that the pumping mechanism 160 can be placed anywhere in relation to the tube 10, as long as the placement is chosen using sound engineering and medical judgment.

The tube 10 may be made of any material chosen in accordance with sound engineering and medical judgment. Preferably, the material should be an inert material, such as silicone, but is not limited thereto.

If the pumping mechanism 160 is provided, it may be activated to aid in the flow of aqueous humor. The method described herein may further comprise the steps of varying pumping mechanism 160 output and achieving desired intraocular pressure according to diurnal fluctuation in aqueous humor production. When a predetermined intraocular pressure is reached, the pumping mechanism 160 output may be decreased. Another step to the foregoing method may include adjusting the pumping mechanism 160 output without having to surgically dissect tissues to expose a large portion of the pumping mechanism 160.

Tubes 10 for drainage of aqueous into aqueous collector channels 44 (probably requiring trabeculotomy or other method of exposing the outer wall of Schlemm's canal), aqueous veins 56, other venous structures in the orbit 114, and larger veins 158 external to the orbit such as the angular vein 158, bypassing any resistance between Schlemm's canal and the venous system. The tubes 10 would be made of silicone or other inert flexible material of appropriate diameter and length depending on whether implantation is planned into the aqueous veins 56, collector channels 44, or more distant venous structures 114, 158. The tube 10 can be coated with heparin, or a similar substance, to prevent clotting of cellular, proteinaceous, fibrinous, and other material draining from the anterior chamber 64.

In another embodiment, the incision can be a full thickness hole at the limbus 153. A substantially straight hole is cut directly through the limbus 153 into the anterior chamber 64 under gonioscopic control. In another embodiment, the tube 10 could be inserted across the anterior chamber 64 through the sclera 124.

1. A method for draining aqueous, the method comprising the steps of:
creating a limbal incision; and,
inserting a drainage tube between an associated anterior chamber and at least one of the group comprising: at least one aqueous vein, at least one collector channel, at least one vein, and at least one distal vein.

2. The method of claim 1, wherein the limbal incision is tunneled from a posterior location in the sclera.

3. The method of claim 1, wherein the limbal incision is a substantially straight hole through the limbus into the anterior chamber.

4. The method of claim 1, wherein creating a limbal incision further comprises the steps of:
creating an incision in the sclera; and,
opening a scleral flap.

5. The method of claim 4, wherein the tube is located on the sclera.

6. The method of claim 5, wherein the method further comprises:
covering the tube with graft material.

7. The method of claim 4, wherein the method further comprises:
covering the tube with the scleral flap.

8. The method of claim 1, wherein the method further comprises the step of:
inserting the drainage tube between the anterior chamber and at least one collector channel.

9. The method of claim 1, wherein the method further comprises the step of:
inserting the drainage tube between the anterior chamber and at least one distal vein.

10. The method of claim 1, wherein the method further comprises the step of:
inserting the drainage tube between the anterior chamber and at least one aqueous vein.

11. The method of claim 1, wherein the method further comprises the step of:
inserting the drainage tube between the anterior chamber and at least one vein.

12. The method of claim 9, wherein the tube is located on the sclera.

13. The method of claim 12, wherein the method further comprises:
covering the tube with graft material.

14. The method of claim 9, wherein the method further comprises:
covering the tube with a scleral flap.

15. The method of claim 1, wherein the method further comprises the step of:
inserting the drainage tube into the anterior chamber with micro forceps.

16. The method of claim 1, wherein the method comprises the step of:
suturing the tube in place via fixation plates and suture holes.

17. The method of claim 1, wherein the method further comprises the step of:
inserting multiple drainage tubes into at least one of the group comprising: multiple aqueous veins, multiple collector channels, multiple veins, and multiple distal veins.

18. The method of claim 1, wherein the method further comprises the steps of:
measuring episcleral venous pressure to select an optimal site for tube placement.

19. The method of claim 18, wherein the episcleral venous pressure is measured using a micromanometer.

20. The method of claim 19, wherein the method further comprises the step of:
coating the tube with an anti-clotting substance.

21. The method of claim 20, wherein the tube is coated with heparin.

22. The method of claim 9, wherein the drainage tube bypasses the aqueous veins and collector channels.

23. The method of claim 1, wherein the method further comprises the step of:
utilizing a lens to magnify the incision.

24. The method of claim 1, wherein the method further comprises the step of:
inserting a pump to ensure that intraocular pressure is maintained.

25. The method of claim 24, wherein the method further comprises the step of:
adjusting the speed of the pump to maintain intraocular pressure.

26. An apparatus for draining aqueous, the apparatus comprising:
a tube, the tube having a proximal and a distal end and a flow opening; and,
at least one retaining device, for holding the tube in place.

27. The apparatus of claim 26, the at least one retaining device comprising:
at least one fixation plate; and,
at least one suture hole.

28. The apparatus of claim 26, the at least one retaining device comprising:
a nipple, the nipple attached to the proximal end.

29. The apparatus of claim 27, the apparatus further comprising:
a one-way valve.

30. The apparatus of claim 29, the apparatus further comprising:
a tapered tip; and,
the nipple being angled at substantially a 135 degree angle with respect to the flow opening.

31. The apparatus of claim 30, the apparatus further comprising:
multiple tubes, the multiple tubes located at the distal end.

32. The apparatus of claim 26, the apparatus further comprising:
a micromanometer.

33. The apparatus of claim 32, the micromanometer comprising:
a transducer;
a bulb;
a wire connecting the transducer to the bulb;
an air intake for the bulb; and,
a pressure sensor.

34. The apparatus of claim 26, the apparatus further comprising:
a tube extender, the tube extender having serrated ridges.

35. The apparatus of claim 26, the apparatus further comprising:
a chuck, the chuck comprising:
a lens;
a port; and,
a channel.

36. The apparatus of claim 26, the apparatus further comprising:
connectors, the connectors having a shape chosen from the group comprising T-shaped and Y-shaped.

37. The apparatus of claim 36, the connectors having serrated ridges.

38. The apparatus of claim 26, the apparatus comprising:
a cowl, the cowl covering the tube;
at least one fixation hole for holding the cowl in place.

39. The apparatus of claim 26, the apparatus comprising:
a pump mechanism, the pump attached the drainage tube.

40. The apparatus of claim 39, the apparatus comprising:
a feedback device, the device connected to the pump.

41. The apparatus of claim 39, wherein the pump mechanism is adapted to draw the aqueous humor from the anterior chamber through the tube and into one of the group comprising: aqueous vein, collector channel, vein, and distal vein.

42. The apparatus of claim 41, wherein the pump mechanism further comprises a power source operatively connected thereto.

43. The apparatus of claim 42, wherein the pump mechanism has length, width, and thickness dimensions of approximately 2 mm by approximately 2 mm by approximately 500 microns, respectively.

44. The apparatus of claim 43, wherein the pump mechanism is implanted posterior to an associated limbus.

45. The apparatus of claim 44, wherein the pump mechanism further comprises a posterior surface, the posterior surface being concave.

46. The apparatus of claim 45, wherein the pump mechanism further comprises a power source having a posterior surface, the posterior surface being concave.

47. The apparatus of claim 46, wherein the pump mechanism has length, width, and thickness dimensions of approximately 6 mm, approximately 10 mm, and approximately 3 mm, respectively.

48. The apparatus of claim 47, wherein the pump mechanism is adapted to operate on a demand basis, such that the required flow through the tube to achieve desired intraocular pressure varies according to diurnal fluctuation in aqueous production.

49. The apparatus of claim 48, further comprising a feedback mechanism for monitoring work performed by the pump mechanism to achieve the desired intraocular pressure.

50. The apparatus of claim 49, wherein the pump mechanism is adapted to be adjusted without having to surgically dissect tissues to expose a large portion of the pumping mechanism.

51. The apparatus of claim 50, further comprising pressure reading means for transmitting intraocular pressure readings to a controller.

52. The apparatus of claim 51, wherein the pump mechanism comprises a wafer.

53. The apparatus of claim 52, wherein the wafer is surrounded by a microchip and an insulating protective layer.

54. The apparatus of claim 53, wherein the pump mechanism is located on the surface of an associated eye.

55. The apparatus of claim 26, the apparatus comprising:
multiple insertion tubes located at the distal end.

56. The apparatus of claim 26, the at least one retaining device comprising:
at least one fixation dagger.
